(19) United States
(12) Patent Application Publication
(10) Pub. No.: US 2024/0417688 A1
(43) Pub. Date: Dec. 19, 2024

(54) METHODS AND SYSTEMS FOR ADIPOCYTES DIFFERENTIATION

(71) Applicants: Steakholder Foods Ltd., Rehovot (IL); The IP Law Firm of Guy Levi, LLC, Wyckoff, NJ (US)

(72) Inventor: Dorit Eliyaev, Rehovot (IL)

(21) Appl. No.: 18/687,833

(22) PCT Filed: Aug. 31, 2022
(86) PCT No.: PCT/US2022/042161

§ 371 Date: Feb. 29, 2024

Related U.S. Application Data
(60) Provisional application No. 63/238,961, filed on Aug. 31, 2021.

(51) Int. Cl. C12N 5/077

(52) U.S. Cl. CPC C12N 5/0653; C12N 2500/36; C12N 2501/33; C12N 2506/1346

(57) ABSTRACT

The disclosure relates to methods, systems and compositions for use in promoting adipocytes differentiation for production of cultured fat ex-vivo. More precisely, the disclosure relates to methods, systems and compositions for use in selectively promoting adipocytes differentiation by exposing MSCs, to plant lecithins for production of cultured fat ex-vivo.

### BACKGROUND

The disclosure is directed to methods, systems and compositions for use in promoting adipocytes differentiation for production of cultured fat ex-vivo. More precisely, the disclosure is directed to methods, systems and compositions for use in selectively promoting adipocytes differentiation by exposing MSCs, to plant lecithins for production of cultured fat ex-vivo.

In an effort to reduce the impact of animal agriculture and to improve people's nutrition, as well as for various other incentives, there is a need for alternatives to animal meat for development of novel protein sources containing viable cells culture(s) that correspond to the three-dimensional (3D) tissue, for instance, muscle tissue and fat tissues.

Fat as a component in cultured meat is essential for acceptance and substantially affect all organoleptic parameters of any food product where it is an integral part.

Therefore, the need exists for methods, systems and compositions for selectively promoting the differentiation of MSCs to adipocytes that can then be used to produce fat ex-vivo.

### SUMMARY

Disclosed, in various exemplary implementations, are methods, systems and compositions for use in selectively promoting adipocytes differentiation by exposing MSCs, to plant lecithin for production of cultured fat ex-vivo.

In an exemplary implementation provided herein is a method of affecting differentiation of Mesenchymal Stem cells (MSCs) to adipocytes comprising admixing a composition comprising vegetable lecithin into a growth medium comprising MSCs, in a concentration (W/V) operable to cause the differentiation of the MSCs to adipocytes.

In another exemplary implementation, provided herein is a composition for differentiating Mesenchymal Stem cells (MSCs) to adipocytes, comprising an effective concentration of vegetable lecithin.

In yet another exemplary implementation, provided herein is an emulsion having a continuous phase and a dispersed comprising the compositions for differentiating MSCs to adipocytes.

### BRIEF DESCRIPTION OF THE FIGURES

For a better understanding of the methods, systems and compositions for use in selectively promoting adipocytes differentiation by exposing MSCs, to plant lecithin for production of cultured fat ex-vivo, with regard to the exemplary implementation(s) thereof, reference is made to the accompanying drawings, in which like numerals designate corresponding elements or sections throughout and in which;

FIG. 1, shows experimental scheme summary;

FIG. 2, depicts 2D cultures stained with Oil Red O for lipid droplet detection after adipogenic differentiation of 5 days. A. negative control culture. B. 4FFAs+Insulin treatment. C. 4FFAs+Insulin+lecithin 1 mg/ml-3 days. D. 4FFAs+Insulin+lecithin 3 mg/ml. E. Insulin+lecithin 1 mg/ml. F. Insulin+lecithin 3 mg/ml; and

FIG. 3, is a graph quantifying the Oil Red O in the various samples.

### DETAILED DESCRIPTION

Provided herein are exemplary implementations of methods, systems and compositions for use in selectively promoting adipocytes differentiation by exposing MSCs, to plant lecithin for production of cultured fat.

Stem cells are classified according to their developmental potential as: (1) totipotent; (2) pluripotent; (3) multipotent; (4) oligopotent; and (5) unipotent. Totipotent cells are able to give rise to all embryonic and extraembryonic cell types. Pluripotent cells are able to give rise to all embryonic cell types. Multipotent cells include those able to give rise to a subset of cell lineages, but all within a particular tissue, organ, or physiological system. For example, hematopoietic stem cells (HSC) can produce progeny that include HSC (self-renewal), blood cell-restricted oligopotent progenitors, and all cell types and elements (e.g., platelets) that are normal components of the blood. Cells that are oligopotent can give rise to a more restricted subset of cell lineages than multipotent stem cells. Cells, which are unipotent, are able to give rise to a single cell lineage (e.g., spermatogenic stem cells). Stem cells are also categorized based on the source from which they are obtained. An adult stem cell is generally a multipotent undifferentiated cell found in tissue comprising multiple differentiated cell types. The adult stem cell can renew itself. Under normal circumstances, it can also differentiate to yield the specialized cell types of the tissue from which it originated, and possibly other tissue types. An embryonic stem cell is a pluripotent cell from the inner cell mass of a blastocyst-stage embryo. A fetal stem cell is one that originates from fetal tissues or membranes. A postpartum stem cell is a multipotent or pluripotent cell that originates substantially from extraembryonic tissue available after birth, namely, the umbilical cord. These cells have been found to possess features characteristic of pluripotent stem cells, including rapid proliferation and the potential for differentiation into many cell lineages. Postpartum stem cells may be blood-derived (e.g., as are those obtained from umbilical cord blood) or non-blood-derived (e.g., as obtained from the non-blood tissues of the umbilical cord and placenta).

Accordingly and in an exemplary implementation, provided herein is a method of affecting differentiation of Mesenchymal Stem cells (MSCs) to adipocytes comprising admixing a composition comprising plant lecithin into a growth medium comprising MSCs, in a concentration (W/V) operable to cause the differentiation of the MSCs to adipocytes.

Plant lecithin is usually defined as a non-uniform mixture of acetone insoluble polar lipids and triglyceride oil, with other minor components produced by water-degumming crude vegetable oils and separating and drying the hydrated gums. Plant lecithins mainly contain phosphatidylcholine (PC), phosphatidylethanolamine (PE), phosphatidylinositol (PI), minor components such as phosphatidic acid (PA), and other substances (e.g., triglycerides, glycolipids, sterols, fatty acids, carbohydrates and sphingolipids). Typical ratios of PC:PE:PI in native plant lecithins are between 15-17 (PC): 6-11 (PE): 10-17 (PI). In an exemplary implementation, the ratio of phosphatidylcholine (PC), phosphatidylinositol (PI) and phosphatidylethanolamine (PE) in the lecithin is non-native, meaning, outside of the above-identified ranges.

The growth medium (referring to a medium useful for culturing cells that promotes growth of cells) used can be, for example, the embryonic stem cell medium, for example, at least one of: a composition comprising: Dulbecco's modified Eagle's medium (DMEM) without Sodium pyruvate having glucose content of between about 70% and about 90%; between about 10% and about 30% Fetal bovine serum (FBS); β-mercaptoethanol (0.1 mM); about 1% of non-essential amino acids; L-Glutamine 2 mM; and basic fibroblast growth factor (BFGF), a composition comprising: Minimum Essential Medium Alpha (MEM-α) with 10% inactivated fetal calf serum, and a composition comprising: DMEM; 15% Fetal bovine serum; Penicillin/Streptomyocin; Glutamine; Non-essential amino acids; nucleosides; β-mercaptoethanol; Sodium pyruvate; and leukaemia inhibitory factor (LIF). Other growth media can be, for example, Ham's F-10+10% fetal calf serum (FCS), Tissue Culture Medium-199 (TCM-199)+10% fetal calf serum, Tyrodes-Albumin-Lactate-Pyruvate (TALP), Dulbecco's Phosphate Buffered Saline (PBS), Eagle's and Whitten's media. For example TCM-199, and 1 to 20% serum supplement including fetal calf serum, newborn serum, estrual cow serum, lamb serum or steer serum. An example of maintenance medium can be TCM-199 with Earl salts, 10% fetal calf serum, 0.2 mM pyruvate and 50 μg/ml gentamicin sulphate. Any of the above may also involve co-culture with a variety of cell types forming a feeder layer such as, at least one of: granulosa cells, oviduct cells, BRL cells, and uterine cells.

Various terms are used to describe cells in culture. “Cell culture” refers generally to cells taken from a living organism and grown under controlled conditions (“in culture” or “cultured”). A “primary cell culture” is a culture of cells, tissues, or organs taken directly from an organism(s) before the first subculture. Cells are “expanded” in culture when they are placed in a growth medium under conditions that facilitate cell growth and/or division, resulting in a larger population of the cells. When cells are expanded in culture, the rate of cell proliferation is sometimes measured by the amount of time needed for the cells to double in number. This is referred to as “doubling time.” Likewise, in the context of the disclosure, the term “differentiation” refers to a process by which a less specialized cell becomes a more specialized cell type. Differentiation is a common process where, for example, stem cells divide and create partially or fully differentiated progeny cells, e.g. during tissue repair and during normal cell turnover. Differentiation dramatically changes a cell's size, shape, membrane potential, metabolic activity, and responsiveness to signals. Furthermore, the term “mesenchymal stem cells” (MSC) denotes multipotent stromal stem cells (in other words, stem cells which are capable of giving rise to many number of cell types) that have the ability to differentiate into cells of the mesodermal lineage, such as adipocytes (fat cells), osteoblasts (bone cells) and chondrocytes (cartilage cells).

In an exemplary implementation, MSCs are obtained from a population of mortal MSCs that may have undergone spontaneous immortalization. In certain exemplary implementations, mesenchymal stem cell cultures typically enter a senescence phase, but are generally able to bypass that phase and continue to divide reaching a crisis phase, with only some sub-populations of these being able to escape from this crisis phase spontaneously, generating the spontaneously-immortalized MSCs (SIMSC's). Additionally, or alternatively, the MSCs are obtained from a population of mortal MSCs that became conditionally immortalized, meaning that the MS cells have a reduced average telomere length over the average telomere length of normal senescent MSCs yet are still capable of unlimited growth, provided the conditionally immortalized MS cells, including but not limited to conditionally immortalized normal MS cells, are maintained in the growth culture conditions, and are periodically transferred to a new growth medium.

In the context of the disclosure, the term “derived” is used to indicate that the cells have been obtained from their biological source and grown or otherwise manipulated in vitro (e.g., cultured in a growth medium to expand the population and/or to produce a cell line). In certain exemplary implementations, the MSCs are derived from at least one of: adipocytes, myocytes fibroblasts, chondrocytes, hepatocytes, umbilical cord blood and umbilical tissue and in the context of the disclosure, refer to pluripotent cells characterized by the properties of, for example, proliferation without transformation, infinite replication, self-renewal and differentiation into all three germ layers; endoderm, mesoderm, and ectoderm. In yet other exemplary implementation, the MSC population is not immortalized.

In an exemplary implementation, the composition comprising vegetable lecithin further comprises phosphate-buffered saline (PBS) solution, and the step of admixing the composition of plant lecithin into the growth medium further comprises emulsifying the lecithin and the PBS solution at a lecithin concentration of between about 1.0 mg/ml and about 3.0 mg/ml, forming a continuous phase and a dispersed phase. The plant lecithin, an amphiphilic compound, will tend to form large micelles that will not be effective to affect cell differentiation. Accordingly and in another exemplary implementation, the lecithin is in the dispersed phase having a volume average (weighted) particle size diameter D_{3,2} of between about 20 nm and 900 nm. Laser diffraction is used in an exemplary implementation for measuring volume weighted distribution and yields the D_{3,2} value and the particle size distribution (PSD). Typically the emulsions disclosed comprise small droplets (the dispersed phase) of the solution of the active substance dispersed in a continuous liquid phase (the continuous phase). The emulsions may be multiple emulsions, for example, a oil-in-water-in-oil (OWO) emulsion. Multiple emulsions may comprise two or more dispersed phases. In a multiple emulsion the solution of the lecithin may be for example, in the internal oil phase. Alternatively, the active substance may be in the aqueous phase. Such multiple emulsions are described by Florence, A. T. and Whitchill, D., (The Formulation and Stability of Multiple Emulsions, Int. J. Pharmaceuticals. 11, 227-308 (1982)). It will be understood that the term ‘continuous phase’ as used herein will, when the emulsion is a multiple emulsion, be taken to refer to the phase which surrounds the dispersed phase comprising the solution of the active substance, even though in a multi-phase emulsion that continuous phase may itself be dispersed. Bicontinuous emulsions, in which both phases are continuous, may be suitable for use in the method disclosed. Where a bicontinuous emulsion is used, it will be understood that the term dispersed phase refers to the phase comprising the lecithin.

In certain exemplary implementations, emulsifying comprises sonicating the mixture for a predetermined period. In other words, the acoustic cavitation phenomenon induced by high intensity ultrasound devices can boost the disruption of lecithin micelles, facilitating the formation of stable emulsions nano emulsions.

In certain exemplary implementations, before, simultaneously, or following the step of admixing the composition comprising plant lecithin into the growth medium, the methods disclosed further comprise admixing a composition comprising a plant-based free fatty acid that is at least one of: palmitoleic, erusic, elaidic, and oleic, into the growth medium, as well as, or alternatively, at least one peroxisome proliferator-activated receptor γ (PPARγ) agonist. The PPARγ agonist is at least one of: thiozolidinedione (TZD), and PPARγ coactivator 1 (PGC-1). For example, TZD, which stimulates 3T3-L1 adipocyte differentiation.

In an embodiment, the methods disclosed are implemented using the compositions described. Accordingly, provided herein is a composition for differentiating Mesenchymal Stem cells (MSCs) to adipocytes, comprising an effective concentration of plant lecithin. Likewise and in yet another exemplary implementation, emulsions of the compositions described are used to affect the differentiation disclosed.

### Example I—Comparing Lecithin to Fatty Acids to Affect Differentiation of MSCs to Adipocytes

### Materials and Methods:

### Free Fatty Acid Preparation

Palmitoleic, erusic, elaidic, and oleic acids are diluted to 20 mM concentration in ethanol absolute. Each FFA is prepared separately, following which, all 6 FFAs are mixed together at equal volumes to create the FFA stock mix.

The stock mix is then added to cell growth medium at a concentration of 100 uM per FFA (dilution of 1:50 from stock mix).

### Lecithin Emulsion Preparation (“Sonic-LE”)

Food grade sunflower lecithin is suspended in PBS 1× at 50 mg/ml concentration, and the suspension is sonicated at medium speed for 1-2 min, to then reach a homogenized solution then filtered through a 0.22 μm filter. The lecithin emulsion is added to cell growth medium at a concentration of 1 mg/ml (dilution of 1:50 from stock solution).

The two mediums were refreshed every 3 days and Oil red O quantification was performed on day 8 of contact.

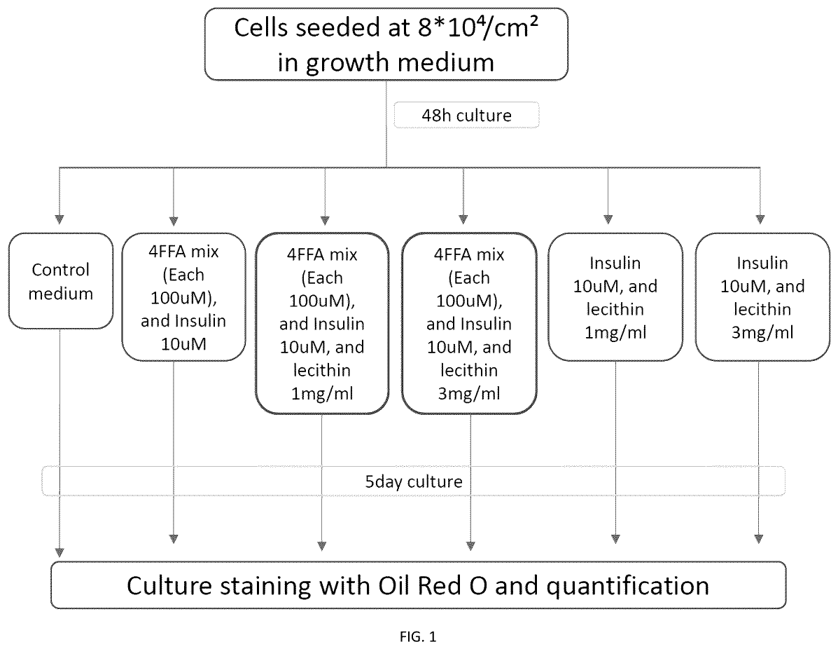

FIG. 1, illustrates experimental scheme used.

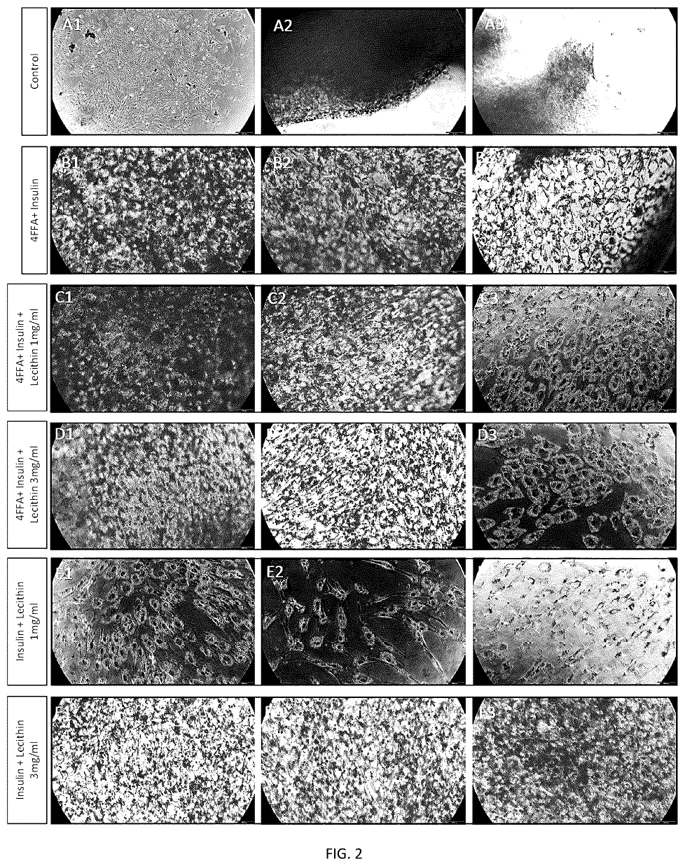

As shown in FIG. 2, although qualitatively there seems to be higher incorporation of the FFAs and differentiation MSCs to adipocytes in the wells exposed to the FFA composition, there is no statistical difference when compared to the biological duplicates exposed to the sonicated lecithin emulsion.

FIG. 2, shows a series of 2D cultures (cultures where cells are not forming clusters or multi-layers) stained with Oil Red O for lipid droplet detection after adipogenic differentiation of 5 days. It is noted, that high cell density can cause a curling effect that produced a cell aggregate, reveling underlying cells. A. (negative control culture)—The cell aggregate falsely stained by ORO (A2); the aggregation reviles few cells on the surface of the well (A1). B. (4FFAs+Insulin treatment) Cells produce a sheet that detaches from the surface (B1), with underlying differentiating cells (B3). C. (4FFAs+Insulin+lecithin 1 mg/ml-3 days) D. (4FFAs+Insulin+lecithin 3 mg/ml). E. (Insulin+lecithin 1 mg/ml). (F. Insulin+lecithin 3 mg/ml).

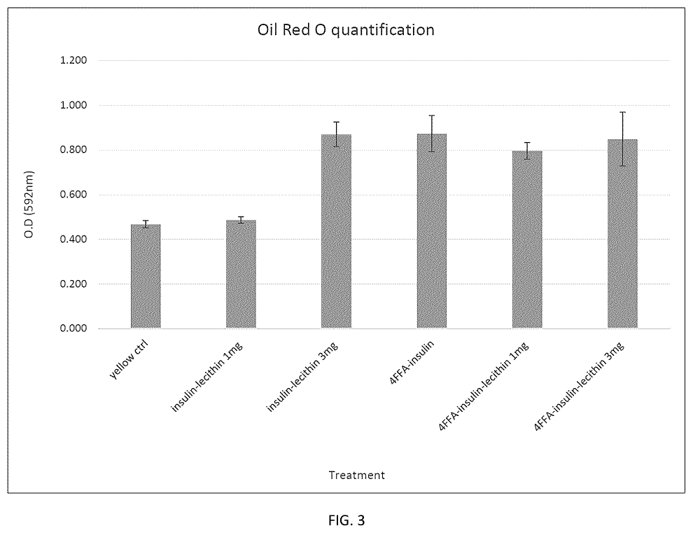

Turning now to FIG. 3, where Oil Red O (ORO) quantification was carried out after extraction using 2-propanol. ORO was extracted and quantified by absorption reading in a plate reader at 492 nm. As illustrated, there is a statistically significant absorption of ORO in all samples comprising the higher dosage of lecithin (3 mg/ml) and insulin and/or the selected four free fatty acids (palmitoleic, erusic, elaidic, and oleic acids).

Conversely, as illustrated in FIG. 3, insulin, a known and potent adipogenic hormone that triggers an induction of a series of transcription factors governing differentiation of pre-adipocytes into mature adipocytes when used alone with the control, shows no statistical difference than control in the amount of ORO quantity, illustrating the lecithin is indeed the dominant differentiation promoter in the composition and the fact that there is no statistical difference in the ORO quantity between samples having the lecithin+4FFA+insulin at the lecithin range disclosed (1-3 mg/ml lecithin), indicates that it is possible to cause differentiation of the derived mortal MSC's without any lecithin.

The term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

All ranges disclosed herein are inclusive of the endpoints, and the endpoints are independently combinable with each other. “Combination” is inclusive of blends, mixtures, alloys, reaction products, and the like. The terms “a”, “an” and “the” herein do not denote a limitation of quantity, and are to be construed to cover both the singular and the plural, unless otherwise indicated herein or clearly contradicted by context. The suffix “(s)” as used herein is intended to include both the singular and the plural of the term that it modifies, thereby including one or more of that term (e.g., the image(s) includes one or more images). Reference throughout the specification to “one exemplary implementation”, “another exemplary implementation”, “an exemplary implementation”, and so forth, when present, means that a particular element (e.g., feature, structure, and/or characteristic) described in connection with the exemplary implementation is included in at least one exemplary implementation described herein, and may or may not be present in other exemplary implementation s. In addition, it is to be understood that the described elements may be combined in any suitable manner in the various exemplary implementations.

All ranges disclosed herein are inclusive of the endpoints, and the endpoints are independently combinable with each other. Furthermore, the terms “first,” “second,” and the like, herein do not denote any order, quantity, or importance, but rather are used to denote one element from another.

Likewise, the term “about” means that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. For example, “about” as used herein mean a reasonable amount of deviation of the modified term such that the end result is not significantly changed. These terms of degree should be construed as including a deviation of at least ±5% or at least ±10% of the modified term if this deviation would not negate the meaning of the word it modifies.

In the context of the disclosure, the term “operable” means the system and/or the device and/or the program, or a certain element or step, or composition is fully functional, sized, adapted and calibrated, comprises elements for, comprises agents and components for and meets applicable operability requirements to perform a recited function when activated, coupled, implemented, actuated, effected, or realized.

Accordingly and in exemplary implementation provided herein is a method of affecting differentiation of Mesenchymal Stem cells (MSCs) to adipocytes comprising admixing a composition comprising vegetable lecithin into a growth medium comprising MSCs, in a concentration (W/V) operable to cause the differentiation of the MSCs to adipocytes, wherein (i) the composition comprising vegetable lecithin also comprises phosphate-buffered saline (PBS) solution, wherein (ii) the step of admixing the composition of vegetable lecithin into the growth medium further comprises emulsifying the lecithin and the PBS solution at a lecithin concentration of between about 1 mg/ml and about 3 mg/ml, forming a continuous phase and a dispersed phase, (iii) wherein the lecithin is in the dispersed phase having an average particle size diameter of between about 20 nm and 900 nm, wherein (iv) emulsifying the emulsion comprises sonicating the mixture for a predetermined period, for example, between about 30 seconds and 1 hour, wherein (v) the MSCs are derived from at least one of: adipocytes, myocytes fibroblasts, chondrocytes, hepatocytes, umbilical cord tissue, and umbilical cord blood, wherein (vi) before, simultaneously, or following the step of admixing the composition comprising vegetable lecithin into the growth medium, the method further comprises a step of admixing a composition comprising a fatty acid of at least one of: palmitoleic, erusic, elaidic, and oleic acids into the growth medium, (vii) the fatty acid further comprises an effective concentration of at least one of: thiozolidinedione (TZD), and PPARγ coactivator 1 (PGC-1), (viii) further comprising a step of admixing insulin, and wherein (ix) the ratio of phosphatidylcholine (PC), phosphatidylinositol (PI) and phosphatidylethanolamine (PE) in the lecithin is non-native, in other words, different from between 15-17 (PC): 6-11 (PE): 10-17 (PI).

In another exemplary implementation, provided herein is a composition for differentiating Mesenchymal Stem cells (MSCs) to adipocytes, comprising an effective concentration of vegetable lecithin, further (x) comprising phosphate-buffered saline (PBS) solution, (xi) at least one of: palmitoleic, erusic, elaidic, and oleic, fatty acids, (xii) at least one peroxisome proliferator-activated receptor γ (PPARγ) agonist, (xiii) the PPARγ agonist is at least one of: thiozolidinedione (TZD), and PPARγ coactivator 1 (PGC-1), wherein (xiv) the ratio of phosphatidylcholine (PC), phosphatidylinositol (PI) and phosphatidylethanolamine (PE) in the lecithin is non-native, in other words, different from between 15-17 (PC): 6-11 (PE): 10-17 (PI).

In yet another exemplary implementation, provided herein is an emulsion having a continuous phase and a dispersed phase comprising the compositions disclosed, wherein (xv) the lecithin is in the dispersed phase having an average particle size diameter of between about 20 nm and 900 nm.

Although the foregoing disclosure for use in selectively promoting adipocytes differentiation by exposing MSCs, to plant lecithin for production of cultured fat ex-vivo, has been described in terms of some exemplary implementations, other exemplary implementations will be apparent to those of ordinary skill in the art from the disclosure herein. Moreover, the described exemplary implementations have been presented by way of example only, and are not intended to limit the scope of the inventions. Indeed, the novel methods, programs, libraries and systems described herein may be embodied in a variety of other forms without departing from the spirit thereof. Accordingly, other combinations, omissions, substitutions and modifications will be apparent to the skilled artisan in view of the disclosure herein.

What is claimed:

1. A method of affecting differentiation of Mesenchymal Stem cells (MSCs) to adipocytes comprising admixing a composition comprising vegetable lecithin into a growth medium comprising MSCs, in a concentration (W/V) operable to cause the differentiation of the MSCs to adipocytes.

2. The method of claim 1, wherein the composition comprising vegetable lecithin further comprises phosphate-buffered saline (PBS) solution.

3. The method of claim 2, wherein the step of admixing the composition of vegetable lecithin into the growth medium further comprises emulsifying the lecithin and the PBS solution at a lecithin concentration of between about 1 mg/ml and about 3 mg/ml, forming a continuous phase and a dispersed phase.

4. The method of claim 3, wherein the lecithin is in the dispersed phase having an average particle size diameter of between about 20 nm and 900 nm.

5. The method of claim 3, wherein emulsifying the emulsion comprises sonicating the mixture for a predetermined period.

6. The method of claim 1, wherein the MSCs are derived from at least one of: adipocytes, myocytes fibroblasts, chondrocytes, hepatocytes, umbilical cord tissue, and umbilical cord blood.

7. The method of claim 1, wherein before, simultaneously, or following the step of admixing the composition comprising vegetable lecithin into the growth medium, the method further comprises a step of admixing a composition comprising a fatty acid of at least one of: palmitoleic, erusic, elaidic, and oleic acids into the growth medium.

8. The method of claim 7, wherein the composition comprising the fatty acid further comprises an effective concentration of at least one of: thiozolidinedione (TZD), and PPARγ coactivator 1 (PGC-1).

9. The method of claim 7, further comprising a step of admixing insulin into the growth medium.

10. The method of claim 1, wherein the ratio of phosphatidylcholine (PC), phosphatidylinositol (PI) and phosphatidylethanolamine (PE) in the lecithin is non-native.

11. A composition for differentiating Mesenchymal Stem cells (MSCs) to adipocytes, comprising an effective concentration of vegetable lecithin.

12. The composition of claim 11, further comprising phosphate-buffered saline (PBS) solution.

13. The composition of claim 11, further comprising at least one of: palmitoleic, erusic, elaidic, and oleic, fatty acids.

14. The composition of claim 11, further comprising at least one peroxisome proliferator-activated receptor γ (PPARγ) agonist.

15. The composition of claim 14, wherein the PPARγ agonist is at least one of: thiozolidinedione (TZD), and PPARγ coactivator 1 (PGC-1).

16. The composition of claim 11, wherein the ratio of phosphatidylcholine (PC), phosphatidylinositol (PI) and phosphatidylethanolamine (PE) in the lecithin is non-native.

17. An emulsion having a continuous phase and a dispersed phase comprising the composition of claim 11.

18. The emulsion of claim 17, wherein the lecithin is in the dispersed phase having an average particle size diameter of between about 20 nm and 900 nm.
